(19) United States
(12) Patent Application Publication
(10) Pub. No.: US 2017/0165101 A1
(43) Pub. Date: Jun. 15, 2017

(54) DEVICE AND METHOD TO ALLEVIATE OBSTRUCTIVE SLEEP APNEA AND/OR SNORING AND/OR INSOMNIA

(71) Applicant: RICHARD DAVIDIAN, New York, NY (US)

(72) Inventor: RICHARD DAVIDIAN, New York, NY (US)

(21) Appl. No.: 14/968,854

(22) Filed: Dec. 14, 2015

(51) Int. Cl. A61F 5/56; A61B 5/00; A61H 23/02; A61M 21/02

(52) U.S. Cl. CPC A61F 5/56; A61M 21/02; A61B 5/4818; A61B 5/4836; A61H 23/02; A61M 2021/0022

(57) ABSTRACT

A device and method to alleviate obstructive sleep apnea and/or snoring and/or insomnia through the use of vibration. The device may be worn in one of several configurations to stimulate the hypoglossal and/or glossopharyngeal nerves, the genioglossus muscle and other muscles of the neck and throat to prevent airway obstruction during sleep. The device may also be worn as a relaxation mechanism that will help alleviate insomnia and promote general relaxation.

### FIELD OF THE INVENTION

This application relates to devices attempting to alleviate obstructive sleep apnea and/or snoring and/or insomnia

### BRIEF DESCRIPTION OF DRAWINGS

FIG. 1: Front view of system and its main components that uses vibration to alleviate snoring and/or OSA and/or insomnia, according to the present invention, worn as a collar around the users neck

FIG. 2: Alternate configuration showing present invention worn as a collar with an external power source and a detection and control module

FIG. 3: Present invention worn in an alternate configuration

FIG. 4: Present invention worn as a headband for alleviating insomnia

### REFERENCE NUMBERS IN DRAWINGS

1: Device of Claim 1
2: Vibrating Elements
3: Switch to control the interval at which vibrations occur
4: Switch to control the amplitude with which vibrations occur
5: Fastening mechanism
6: Internal battery, which may be removable and/or rechargeable
7: Detection and control module
8: Connecting cords from Device of Claim 1 to detection and control module and external battery source
9: External battery source
10: Power cord from external battery to outlet

### BACKGROUND OF THE INVENTION

Obstructive Sleep Apnea (OSA) is a problem that has an adverse effect on many individuals. It is typically caused by a lack of adequate spacing in the back of the oral cavity. As one sleeps, the relaxation of muscles and soft tissue in the back of the oral cavity causes the spacing to collapse, preventing proper airflow and causing apneas during which the individual is not breathing properly. As we age, the tissue and muscle in the oral cavity continue to weaken, reducing the spacing further and leading to a higher likelihood of OSA. OSA can ultimately affect overall health, as lack of proper sleep can lead to many other health issues.

Snoring is a common problem for many people. Snoring is caused by a partial occlusion of the breathing space, as distinguished from OSA, which is a full occlusion for short periods of time. Snoring by itself is not as injurious to ones health as OSA. It may, however, also impact the sleep of companions.

Insomnia is difficulty in falling asleep, or falling back to sleep once awakened. In more severe cases, the lack of sleep it causes can lead to further health complications.

There have been many attempts at device solutions to OSA in the Prior Art. The most common is CPAP (continuous positive airway pressure), which can be an effective solution for people with moderate to severe sleep apnea [e.g. Sullivan U.S. Pat. No. 7,141,021]. However, the disadvantages to CPAP are numerous. A CPAP machine is restrictive to sleep positions due to tubing and mask that tether the user to the CPAP machine. It is unattractive and cumbersome to wear. It can dry out the mucosa. It requires a difficult adaptation period for the user to adjust to being able to sleep with constant air pressure. It is not easily portable when the user travels. It can be noisy. It requires an outlet nearby. It is relatively expensive. It requires ongoing maintenance. For these reasons and others CPAP is generally an uncomfortable and not very flexible design solution. This is born out in the daily usage rates, which have been estimated at approximately 50%. CPAP effectiveness also depends on factors such as irritation in nasal and oral passageways due to allergies, illnesses, smoking, etc. and as such may not always provide a consistent solution given many users have fixed settings from their doctors/providers that they are not allowed to change on their own. Finally there are some health risks including but not limited to: too high a pressure may overinflate the lungs; improper cleaning can lead to respiratory infections. Other device solutions are those that maintain the tongue positioned forward while sleeping [e.g. Dort U.S. Pat. No. 7,946,292].

The disadvantages here are that it becomes hard to swallow, makes drooling likely, dries out and can irritate the tongue overnight, requires regular cleaning maintenance, and again is generally not comfortable as an everyday usage solution. Another group of device solutions are those oral appliances that either move the lower jaw forward, attempt to hold the tongue down, or both [e.g. Halstrom U.S. Pat. No. 6,729,335]. These have the disadvantage of potential irritation of the jaw over long periods of usage, negative impact on proper bite mechanics and a lack of general comfort leading to potentially low compliance. Finally, there are some devices that send electrical shocks in an attempt to stimulate muscles to keep the airway open [e.g. U.S. Pat. No. 9,114,256]. These have the disadvantage that depending on individual sensitivities, electrical shocks may not be comfortable during sleep, leading to potentially low compliance, and it is not clear what long term effects electrical stimulation may have on the long term proper firing of nerves.

There are also many surgical solutions. Uvulopalatopharyngoplasty (UPPP) is one that removes soft tissue from the soft palate to create more space. Long-term results have been mixed, with 50% success rates and sometimes worsening problems over time as scar tissue weakens. Another is implantable electronic circuitry designed to stimulate the hypoglossal nerve, the genioglossus muscle and other such muscles, whose main function is to move the tongue forward [e.g. Bolea U.S. Pat. No. 9,186,511]. During deeper sleep, these muscles relax and allow the tongue to slip back (particularly when sleeping in supine position) and occlude the breathing airway. The electronic stimulation is intended to get the muscles to fire to keep the airway open. As with the external device version of this solution mentioned above, it is not clear that this will be comfortable to all individuals during sleep nor what impact it will have on proper nerve function over the long term. Finally, all surgical solutions put the patient at risk during surgery, may have high costs associated with it, require the higher commitment level of the patient to be willing to live with something implanted in their bodies, and/or the irreversibility of having tissue removed.

### BRIEF DESCRIPTION OF THE INVENTION

The present invention is a device that delivers vibration to the relevant muscles of the pharynx and larynx and/or stimulates the glossopharyngeal and hypoglossal nerves to alleviate snoring and/or OSA. It can also be used to deliver vibration to alleviate insomnia. Obstructive sleep apnea and snoring occur in many cases because of a relaxation of the muscles and soft tissue of the oral cavity and/or a falling back of the tongue in the mouth that leads to full or partial blockage, respectively, of the air passageway that must remain open to allow for breathing. The problem is usually exacerbated when the user sleeps on his/her back, as the force of gravity is stronger in acting on the tongue, uvula, and other aspects of the soft palate. When users enter deep sleep, sometimes referred to as Stage 3 sleep, and REM sleep, the firing of the genioglossus muscle to pull the tongue forward so as not to block the airway does not occur as when in the awakened state. In general during deep sleep, muscles, except those controlled by the central nervous system, are fully relaxed and lead to a narrowing of the airway, which under inspiration can partially obstruct and vibrate (snoring) or fully obstruct (OSA). The list of ways the device being described herein alleviates snoring and/or obstructive sleep apnea and allows the user to follow a normal sleep cycle includes but is not limited to the following: using vibration to perturb the muscles of the pharynx and/or larynx such that they never fully relax and thereby obstruct the airway; using vibration placed at specific locations as preset in the device to stimulate the hypoglossal nerve, which controls the functioning of the tongue, and/or the glossopharyngeal nerve, which also impacts the tongue, such that the tongue does not obstruct the airway; using vibration to prevent the genioglossus muscle from fully relaxing and thereby preventing the tongue from falling back fully and obstructing the airway.

For insomnia, often the problem occurs because individuals can not clear their mind from thoughts of the day that prevent one from entering a sleep state. Solutions such as “counting sheep” to distract one's attention from troublesome thoughts are well known, but often not effective. By placing the device described herein around the forehead, as shown in FIG. 4, the relaxing effect of the vibration will distract the user and allow for sleep to occur. As one approaches sleep, the user can remove the device if so desired.

Compared to all surgical procedures that currently exist for snoring and OSA, the advantages of the device described herein are that it is non-invasive, is reversible, requires a lower commitment than undergoing surgery, is low cost and has lower risk.

Compared to CPAP, which is arguably the most common device solution, the device described herein is more comfortable, less restrictive, doesn't dry out the mucosa, will not cause respiratory infections, is user settable and will lead to higher long term compliance rates for all of these reasons. It is also more portable, less noisy, does not require maintenance, and is relatively inexpensive. The user, in general, is less inconvenienced and more comfortable and can interact with their partners as they ordinarily would, thus improving quality of life. Compared to oral devices that either move the lower mandible forward or hold the tongue out physically, the device herein does not have to be worn inside the mouth, and therefore is more comfortable. It will not lead to jaw pain over extended use. It will not impact bite mechanics. It will not create issues with swallowing properly during sleep. There will be no drooling. The user, in general, is less inconvenienced and more comfortable and can interact with their partners as they ordinarily would, thus improving quality of life. Compared to electrical stimulation devices, it is a gentler solution and therefore more likely to allow for uninterrupted sleep, is lower risk and more comfortable to use, leading to higher compliance rates.

### DETAILED DESCRIPTION OF INVENTION

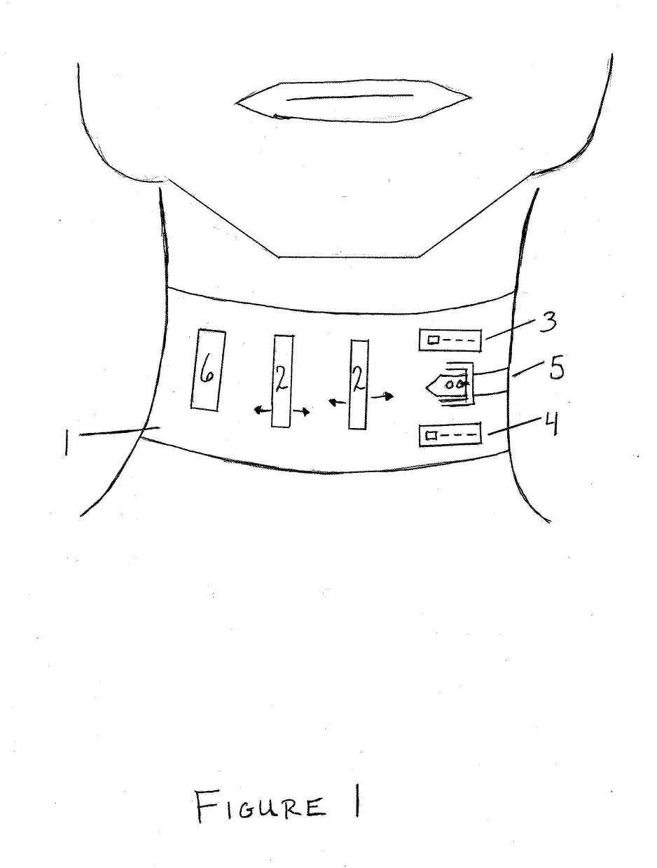

One embodiment of the device is shown in FIG. 1. It consists of a collar [1] that contains one or more vibrating elements [2] to be worn around the neck of the user. The vibrating elements may be movable into different slots in the device to optimize their effectiveness. In the embodiment shown in FIG. 1, two vibrating elements are shown [2]. There are two multi-setting switches, one for setting the interval with which vibration occurs [3] and one for setting the amplitude of vibration [4]. In other embodiments, these switches may be dials or some other mechanism for the user to select amongst various settings. In this embodiment, a fastening mechanism [5] is used to size the collar. In other embodiments, the collars will be sized to fit the user, or be of a stretchable material to accommodate different user sizes, and thus no fastening mechanism will be present. The device will have it's own battery source [6], which may be removable and/or rechargeable.

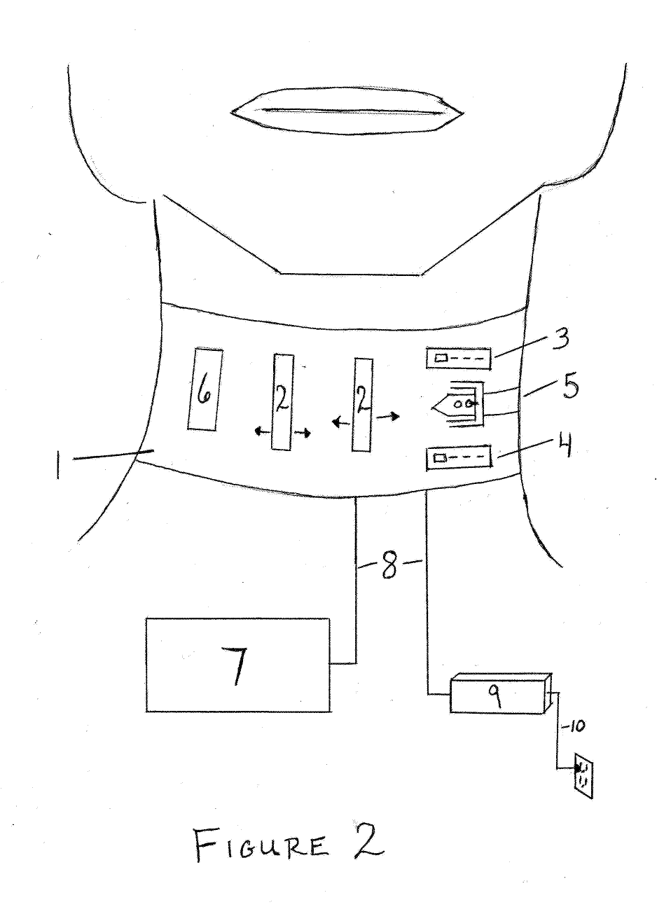

FIG. 2 shows the device in another embodiment, with a detection and control module [7]. This module will make it such that vibration only occurs when snoring and/or obstructions are detected to be occurring. The advantage of this approach would be to enhance battery life if a battery is employed, to use less power overall, and to be potentially less disruptive. In this embodiment, there is also an external power supply [9] that may be detachable from the collar device [1]. The external power supply may consist of a rechargeable battery, or be plugged directly to an electrical outlet with a power cord [10]. The detection and control module [7] and the external power supply [9] will each have a long enough connecting cord [8] to the device [1] such that they could be placed on a bedside table if the user prefers this arrangement.

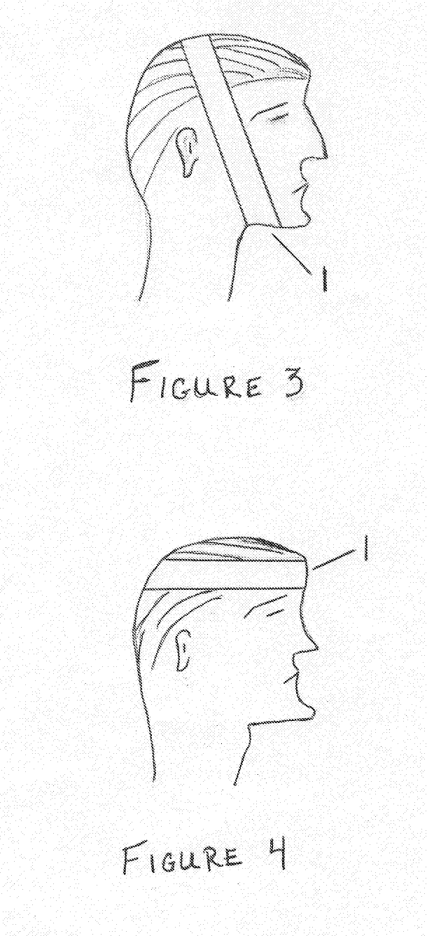

FIGS. 3 and 4 show alternative configurations of wearing the device [1] to the configuration shown in FIGS. 1 and 2, depending on which is most effective and comfortable for the user given the type of problem the user is trying to solve and the users own physiognomy. FIG. 3 is an alternative configuration for alleviating OSA and snoring. FIG. 4 shows the preferred configuration for dealing with insomnia.

The foregoing description of certain preferred embodiments and configurations is set forth for the purpose of illustrating the principals of the invention. Since numerous alternative uses, modifications, configurations and changes will readily occur to those skilled in the art, it is not desired to limit the invention to the exact construction and operation shown and described above, in the drawings and within the claims. Thus, all suitable modifications, configurations and equivalents and alternate applications will fall within the scope of the present invention, which is defined by the claims herein.

What is claimed is:

1. A device to alleviate sleep apnea and/or snoring and/or insomnia through the use of vibration, comprising:
a collar to be worn, adjustable in size by some fastening mechanism, or of material that may be stretchable to accommodate variation in sizing,
said collar containing a means of vibration, which may be user settable to be either continuous or to occur at one of multiple settings of differing fixed intervals, and/or may be user settable to occur at one of multiple settings of differing fixed and/or varying amplitudes of vibration, whose action will prevent relaxation of the muscles, tissues and/or organs that leads to collapse of the airway and resultant snoring and/or obstructive sleep apnea, either through perturbations created by said vibration preventing said muscles, tissues and organs from fully relaxing or by stimulating the hypoglossal and/or glossopharyngeal nerves or by stimulation of appropriate muscular action, whether the genioglossus muscle or other, by nerve firing or some other means
a power source to power said means of vibration, that may be either a battery source, rechargeable or not, detachable or not, contained in said collar such that the device is completely self-contained, or an external power source that may be a battery or connection directly to electrical power source, that may be permanently attached to or detachable from said collar.

2. The device of claim 1, worn around the neck

3. The device of claim 1, worn as a headband around the head to alleviate insomnia

4. The device of claim 1, worn under the chin and over the head in any one of varying angles

5. The device of claim 1, using a buckle as the fastening mechanism

6. The device of claim 1, using Velcro as the fastening mechanism

7. The device of claim 1, coming in different sizes to accommodate different neck and head sizes

8. The device of claim 1, using a passive power source

9. The device of claim 1, further comprising a means of detection to determine when apneas and/or hypopneas and/or snoring are occurring and a means of control to only vibrate in response to such occurrences

10. The device of claim 1, used to promote relaxation

11. A method of using vibration to alleviate insomnia or to prevent muscles and soft tissue in the oral cavity from relaxing to the point of occluding breathing, thereby reducing OSA and snoring, comprising the steps of:
a. Selecting an appropriate device to supply vibration
b. Setting the desired frequency of vibration
c. Setting the desired amplitude of vibration
d. Wearing the device during sleeping hours to alleviate snoring and/or obstructive sleep apnea and/or insomnia

12. A method of using vibration to exercise and tone the muscles of the neck and throat such that they become strong enough on their own to maintain proper airflow when sleeping, comprising the steps of:
a. Selecting an appropriate device to supply vibration
b. Setting the desired frequency of vibration
c. Setting the desired amplitude of vibration
d. Wearing the device when asleep or awake on a regular basis, for enough time such that the muscles of the neck and throat are exercised enough to maintain proper tone when sleeping without the device and proper air intake occurs without obstruction

13. The device of claim 1, used to alleviate insomnia
